(19) United States
(12) Patent Application Publication
(10) Pub. No.: US 2011/0054317 A1
(43) Pub. Date: Mar. 3, 2011

(54) TRACKING AND OPTIMIZING GAIN AND CONTRAST IN REAL-TIME FOR ULTRASOUND IMAGING

(76) Inventors: Feng Lin, Niskayuna, NY (US); Suhyun Park, Schenectady, NY (US); Xiaodong Tao, Niskayuna, NY (US); Mirsaid Seyed-Bolorforosh, Lake Forest, IL (US); Kai Thomenius, Clifton Park, NY (US); Menachem Halmann, Milwaukee, WI (US)

(21) Appl. No.: 12/551,206

(22) Filed: Aug. 31, 2009

(51) Int. Cl. A61B 8/14; G06K 9/00

(52) U.S. Cl. 600/443; 382/131

(57) ABSTRACT

A system for dynamic optimization of gain and contrast in ultrasound imaging includes an image processor module programmed to dynamically estimate a correction profile in real-time and apply the correction profile to adjust a gain and contrast of image frame data sets. The image processor module is programmed to identify tissue and background regions in an image frame data set, determine an image intensity for each of the tissue and background regions, and formulate a gain profile based on the image intensity of the tissue region to compensate the gain variation of an image. The image processor module is further programmed to calculate an image contrast metric based on the image intensity of the tissue and background regions, and modify a gray map of the image frame data set based on the image contrast metric to adjust the contrast of an image displayed on the display system.

### BACKGROUND OF THE INVENTION

The invention relates generally to a system and method for ultrasound imaging and, more particularly, to a system and method for dynamic optimization of gain and contrast in a real-time fashion for ultrasound imaging.

As is well known, in ultrasound imaging, a series of high-frequency sonic pulses are generated, and these pulses “bounce” off various objects in their path. Specifically, different structures in a patient's body exhibit different levels of impedance, and ultrasound echoes are generated when the ultrasound signals contact impedance boundaries between these structures. The interval between the emission of the pulses and the receipt of the corresponding echoes is measured to determine the distance between the source of the pulse and the impedance boundary from which the echo resulted. In addition, the relative intensity of the echo conveys information regarding the nature of the tissues causing the echoes. Different tissues exhibit different levels of impedance to the ultrasound signals. Therefore, varying impedance differentials exist, for example, at the boundary between muscle tissue and bone as opposed to the boundary between fatty tissue and organ tissue. As a result, when an ultrasound pulse strikes the impedance boundary between muscle tissue and bone, a more robust echo is generated than the echo generated when an ultrasound pulse strikes the impedance boundary between fatty tissue and organ tissue. Ultimately, it is the mosaic assembled from each of these echoes received, reflecting the position and the nature of the objects causing the echoes, that constitutes the multi-dimensional images obtained through the use of ultrasound imaging.

In ultrasound imaging, there are a number of issues that must be addressed in order for the ultrasound system to generate a useful image having sufficient resolution to help a medical professional assess the portion of the patient's body being studied. One such issue relates to the attenuation of ultrasound signals as they penetrate into a region sought to be imaged. In B-mode ultrasound imaging, two-dimensional images of tissue are created in which the brightness of a pixel is based on the intensity of the echo return. During conventional two-dimensional imaging, gain adjustments provide overall image changes. The gain is typically adjusted after beamforming and before image processing, i.e., prior to envelope detection. Gain adjustment in the axial direction, known as “time gain compensation” (TGC), is carried out by increasing or decreasing gain as a function of depth. In addition, “lateral gain compensation” (LGC) can be used to adjust the gain setting as a function of lateral position.

The TGC block at the output of the beamformer is basically a depth-dependent gain control designed to compensate the received signal to correct for the attenuation caused by tissues at increasing depths. It is often set based on a nominal tissue attenuation factor (e.g., 0.5 dB/cm-MHz) and beam diffraction losses as a function of depth. The objective is to produce uniform tissue image brightness from the near field to the far field. In practice, the tissue attenuation properties may deviate from the assumed constant factor (or an application-dependent internal TGC curve), and may vary significantly with depth, especially if macroscopic structures and reflectors are present. Further, if the far-field regions are very noisy, it is desirable to suppress their pixel intensities for best overall image presentation. For these reasons, manual TGC adjustment is usually provided via a column of sliding controls (potentiometers) or rotary knobs on the front panel, for different depth zones. That is, a manual control mode is implemented, where a set gain is applied until the user manually adjusts the setting.

Since then, static-type automatic gain compensation methods have been reported. In those methods, the user is required to click a button to activate the gain compensation process. Once the button is clicked, the scanner automatically analyzes the tissue properties and generates a correctional gain and TGC/LGC profile, which is applied to subsequently frames. This is a “static” control mode where user intervention is needed and the user needs to click the button again once the targeted tissue is changed and the image gain is not optimal anymore.

Recently, attempts to design scanners that automate gain compensation have been undertaken. Such scanners oversee gain change over time and, if the difference is within a threshold, the scanner will still use the original correction profile as does in static optimization. If the difference is over a threshold, it activates another optimization process and the gain profile is updated once. While preferable to the static optimization, these existing “semi-dynamic” compensation techniques may result in users experiencing sudden unexpected gain changes based on a delayed gain adjustment, which is undesirable. Thus, a need exists for an improved system capable of providing real-time, smooth automated gain compensation based on actual image data.

Another issue in generating a useful ultrasound image is that of noise. In any system, the signal of interest subsists against a background of ambient signals. These other signals have nothing to do with the signal of interest, other than the fact that these unwanted signals interfere with the signal of interest. These ambient signals constitute a noise component. Furthermore, some external noise sources, such as electrical equipment used proximally to the ultrasound imaging equipment, may introduce noise into the signal path. In addition, as a result of electrons moving through the components of the imaging system itself, the imaging system will exhibit thermal noise.

Both the noise and the desired signals are detected by the ultrasound imaging system. Unfortunately, if the noise were to be analyzed as though it were part of the desired signal, the resulting ultrasound image would be compromised, and the image then would inaccurately portray structures in the patient's anatomy. To avoid the noise unduly compromising the integrity of the desired signals, the desired signals must be separated from the noise, or the noise must be suppressed as much as practical. One way to preserve the integrity of the signal, when the magnitude of the desired signals exceeds that of the noise, is to adjust the dynamic range used in mapping the signal to the display of the system through the use of a mapping function referred to as a compression map. As is well known, the dynamic range is an expression of the ratio of the received magnitudes of the largest signal to that of the smallest discernible signal. Dynamic range also commonly known as the signal-to-noise ratio. Reducing or compressing the dynamic range, in effect, involves cutting off signals having a magnitude below a predetermined value. The dynamic range can be compressed by programming the signal/image processing unit to disregard signals having a magnitude below a certain level so that these unwanted signals do not unduly compromise images shown on the display. If the magnitude of the useful component of the signals conveying information about the patient's tissues is largely greater than the magnitude of the noise component, the noise component can be partially or completely suppressed, leaving a useful signal largely free of noise from which an image can be derived.

However, if the dynamic range is compressed too much, it detracts from the ability to process desired signals. The problem is relatively insignificant when there is relatively little noise present, because suppressing lower magnitude noise signals still allows the system to process desired signals over a larger useable dynamic range. However, as the magnitude of the noise increases, desirable signals will be increasingly affected and “drowned out” by the noise. As the dynamic range is increasingly compressed to avoid the increased noise, unfortunately, desirable signals having magnitudes comparable to the magnitude of the noise also will be lost.

In adjusting the dynamic range, histogram specifications can be employed as an image enhancement technique to modify the image so that it has a particular contrast. Multiple histogram equalization or specification methods have been previously been developed, such as a general histogram specification method. However, such a method does not differentiate between regions of noise, tissue, or bright interface and hence the performance is affected by relative sizes of different regions. Dynamic range enhancement methods have also been described that adjust gray scale based on the distributions of lowest gray levels and highest gray levels. However, such methods do not analyze the center portion of the histogram where tissue information resides and hence the performance may be limited. Still another histogram method previously developed is an analytical method that decomposes the overall histogram into noise, tissue, and bright interface distributions and determines the contrast correction based on the three distributions. However, the analytical method assumes standard distributions of the three regions and thus the decomposition is prone to error. Robust methods to separate tissue, noise, and bright interface are thus needed.

It would therefore be desirable to have a system and method capable of providing real-time, smooth automated gain compensation based on actual image data. It would further be desirable to have a system and method capable of real-time tracking and correction of dynamic range to provide for automatic contrast adjustment, with the dynamic range tracking and correction being based on tissue segmentation to reliably estimate and adjust image contrast for different clinical applications.

### BRIEF DESCRIPTION OF THE INVENTION

The invention provides a system and method for dynamic optimization of gain and contrast in a real-time fashion for ultrasound imaging.

In accordance with one aspect of the invention, an ultrasound system includes a transducer array comprising a multiplicity of transducer elements configured to acquire image frame data sets of raw acoustic data samples during ultrasound scanning of an imaging volume and a display system for displaying an image frame data set. The ultrasound system also includes an image processor module programmed to dynamically estimate a correction profile in real-time and apply the correction profile to adjust a gain and contrast of the image frame data sets. In being programmed to dynamically estimate the correction profile, the image processor module is programmed to identify a tissue region and a background region in an image frame data set, determine an image intensity for each of the tissue region and the background region, and formulate a gain profile based on the image intensity of the tissue region to compensate the gain variation of an image displayed on the display system. The image processor module is further programmed to calculate an image contrast metric based on the image intensity of the tissue region and the image intensity of the background region and modify a gray map of the image frame data set based on the image contrast metric to adjust the contrast of an image displayed on the display system.

In accordance with another aspect of the invention, a method for dynamically optimizing gain and contrast in ultrasound imaging during acquisition of a plurality of frames of ultrasound image data includes the steps of segmenting a frame of ultrasound image data into a plurality of tissue blocks and a plurality of non-tissue blocks and determining an image intensity for each of the plurality of tissue blocks and each of the plurality of non-tissue blocks. The method further includes the steps of estimating a gain correction for the frame of ultrasound image based on the segmented plurality of tissue blocks and the plurality of non-tissue blocks, estimating a contrast correction based on the image intensity of the plurality of tissue blocks and the image intensity of the plurality of non-tissue blocks, and applying a correction profile to the frame of ultrasound image data based on the estimated gain correction and the estimated contrast correction.

In accordance with yet another aspect of the invention, an ultrasound system includes a transducer array comprising a multiplicity of transducer elements configured to acquire frames of ultrasound image data during ultrasound scanning of an imaging volume. The ultrasound system also includes an image processor module operationally connected to the transducer array to receive the frames of ultrasound image data therefrom, wherein the image processor module is programmed to: (a) segment a first frame of ultrasound image data into a plurality of tissue blocks and a plurality of non-tissue blocks, (b) determine an image intensity for each of the plurality of tissue blocks and each of the plurality of non-tissue blocks, and (c) estimate a gain correction for the first frame of ultrasound image based on the segmented plurality of tissue blocks and the plurality of non-tissue blocks. The image processor module is further programmed to: (d) estimate a contrast correction for the first frame of ultrasound image based on the image intensity of the plurality of tissue blocks and the image intensity of the plurality of non-tissue blocks, (e) apply the estimated gain correction and the estimated contrast correction to the first frame of ultrasound image data, and (f) estimate a modified gain correction and a modified contrast correction for a next frame of ultrasound image data according to steps (a)-(d), the modified gain correction and the modified contrast correction being applied to the next frame of ultrasound image data.

Various other features and advantages will be made apparent from the following detailed description and the drawings.

### BRIEF DESCRIPTION OF THE DRAWINGS

The drawings illustrate preferred embodiments presently contemplated for carrying out the invention.

In the drawings:

FIG. 1 is a schematic block diagram of an ultrasound system according to an embodiment of the invention.

FIG. 2 is a block diagram illustrating a process for dynamic optimization of gain and contrast for use with the ultrasound system of FIG. 1 according to an embodiment of the invention.

FIG. 3 is a block diagram illustrating an image data compression algorithm for use in the process of FIG. 2 according to an embodiment of the invention.

FIG. 4 is a block diagram illustrating an estimation algorithm for use in the process of FIG. 2 according to an embodiment of the invention.

FIG. 5 is an illustration of an adaptive slope change method for dynamic contrast estimation for use in the estimation algorithm of FIG. 4 according to an embodiment of the invention.

FIG. 6 is an illustration of tissue specific histogram specification method for dynamic contrast estimation for use in the estimation algorithm of FIG. 4 according to an embodiment of the invention.

FIG. 7 is a block diagram illustrating a compensation/correction algorithm for use in the process of FIG. 2 according to an embodiment of the invention.

FIG. 8 is a block diagram illustrating the gain and contrast estimation and compensation/correction updating process according for in the process of FIG. 2 according to an embodiment of the invention.

### DETAILED DESCRIPTION

According to embodiments of the invention, an ultrasound system is provided that functions to dynamically optimize gain and contrast in a real-time fashion during ultrasound imaging. The ultrasound system enables point-and-shoot ultrasound imaging, without the need for an operator to manually adjust gain and contrast settings, thereby freeing up an operator to focus on medical diagnosis and helping improve workflow.

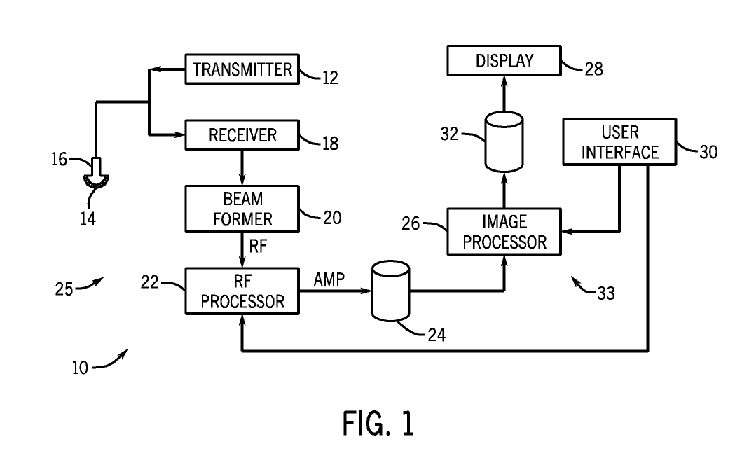

According to an embodiment of the invention, FIG. 1 illustrates an ultrasound system 10 including a transmitter 12 that drives an array of elements 14 (i.e., transducer elements) within an ultrasound transducer 16 to emit pulsed ultrasonic signals into a body or imaging volume. The elements 14 may be arranged, for example, in one or two dimensions. Each ultrasound transducer 16 has a defined center operating frequency and bandwidth. The ultrasonic signals are back-scattered from structures in the body, like fatty tissue or muscular tissue, to produce echoes that return to the elements 14. The echoes are received by a receiver 18 and are passed through beam-forming electronics 20 to acquire image data from the raw acoustic data received by ultrasound transducer 16. Beam-forming electronics 20 perform a beam-forming function and output an RF signal, which then passes through an RF processor 22. The RF processor 22 may include a complex demodulator (not shown) that demodulates the RF signal to form IQ data pairs representative of the echo signals. It may also include a gain and TGC/LGC control unit to adjust the signal amplitude. The RF signal or IQ data pairs may further be filtered, decimated, envelope detected, and compressed to form compressed envelope data. The image frame data sets (i.e., image data) are then routed to a memory 24 for storage or directly to an image processor module 26, according to embodiments of the invention. As shown in FIG. 1, the components 12-22 form front-end hardware 25.

According to embodiments of the invention, image processor module 26 is configured to process the acquired ultrasound information (i.e., image frame data sets) and prepare frames of ultrasound information for display on display 28. Acquired ultrasound information may be processed and displayed in real-time during a scanning session as the echo signals are received. Additionally or alternatively, the ultrasound information may be stored temporarily in memory 24 during a scanning session and then processed and displayed in an off-line operation.

The processor module 26 is connected to a user interface 30 that may control operation of the processor module 26 as explained below in more detail. The display 28 includes one or more monitors that present patient information, including diagnostic ultrasound images to the user for diagnosis and analysis. One or both of memory 24 and memory 32 may store three-dimensional (3D) data sets of the ultrasound data, where such 3D datasets are accessed to present 2D and 3D images. Multiple consecutive 3D datasets may also be acquired and stored over time, such as to provide real-time 3D or 4D display. The images may be modified and the display settings of the display 28 also manually adjusted using the user interface 30. As shown in FIG. 1, the components 24-32 collectively form back-end electronics 33.

According to an exemplary embodiment of the invention, image processor module 26 is programmed to dynamically adjust gain and contrast in ultrasound system 10 to provide for formation of an optimal image on display 28. That is, as opposed to prior art systems with manual gain and contrast adjustment and/or static automatic adjustment, image processor module 26 of ultrasound system 10 is programmed to automatically and dynamically adjust gain and contrast for optimal generation of an image on display 28. Such dynamic adjustment by image processor module 26 enables point-and-shoot ultrasound imaging, without the need for an operator to manually adjust settings on ultrasound system 10. Ultrasound system 10 thus frees up an operator to focus on medical diagnosis and helps improve workflow, negating the requirement for manual adjustment of gain and contrast settings.

According to embodiments of the invention, image processor module 26 functions to perform time gain compensation (TGC) to fine tune the image in the axial direction by increasing or decreasing gain as a function of depth (time) for all received vectors. Image processor module 26 can also function to perform lateral gain compensation to fine tune the image in the lateral direction by increasing or decreasing gain as a function of lateral position (beam or vector position). In the former case, gain is controlled in small rows of the image. In the latter case, gain is controlled in small sectors of the image. In the case where a two-dimensional gain compensation is provided, a respective gain can be automatically applied to each point along a vector, the gain being a function of depth and vector angle.

Image processor module 26 also functions to perform dynamic range (i.e., contrast) compensation. According to embodiments of the invention, the contrast of the ultrasound images is adjusted by creating gray maps, which are simple transfer functions of the raw acoustic sample data to display gray values. Image frame data sets are received by image processor module 26, which generates a gray map based on one or more image frame data sets and in which the range of acoustic sample values are correlated to a gray-scale value range from a minimum gray scale value (e.g., 0) to a maximum (e.g., 255) gray scale value. The acoustic sample values outside the new gray map input range are mapped to the minimum or the maximum. According to an exemplary embodiment of the invention, image processor module 26 functions to optimize the contrast by dynamically compensating or correcting the gray map of an image frame data set, as is set forth in further detail below.

According to an embodiment of the invention, the gain, TGC/LGC, and dynamic range adjustments may also be made in RF processor module 22 (in front-end hardware 25) through the RF filtering process and the compression process. The processes are typically done in hardware like an application specific integrated circuit (ASIC) or field-programmable gate array (FPGA), or programmable devices like digital signal processors (DSP).

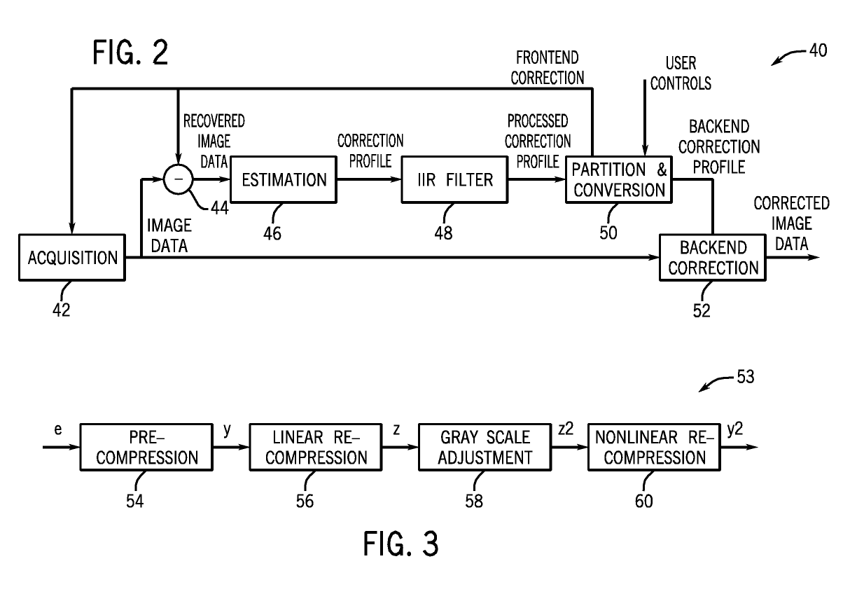

Referring now to FIG. 2, a schematic block diagram illustrating a processor-implemented technique 40 for dynamic optimization of gain and contrast is shown, such as can be implemented by image processor module 26 (and RF processor module 22). At BLOCK 42, data is acquired in a known manner by way of an ultrasound system, such as the ultrasound system 10 illustrated in FIG. 1, for example. At BLOCK 44, the effect of front-end gain and contrast compensation (such as performed by RF processor 22 of FIG. 1) is removed to recover the original image data. Next, at BLOCK 46, image data from the acquisition is analyzed in an estimation block to generate a correction profile (i.e., compensation profile) for gain and dynamic range that is applied to acquired image frame data sets (i.e., image data). At BLOCK 48, the correction profile generated from BLOCK 46 is processed by a filter (e.g., an infinite impulse response filter or IIR filter) to output a processed correction profile. The processed correction profile is then applied to the front-end hardware 25 and/or the back-end software 33 of ultrasound system 10 (FIG. 1). As shown in FIG. 2, a partition block, BLOCK 50, divides the correction into front-end and back-end parts and sends the correction profile to acquisition block, BLOCK 42, and backend correction block, BLOCK 52, respectively. According to one embodiment of the invention, user controls may also be fed into the partition block, BLOCK 50, (via user interface 30 of FIG. 1, for example) to apply extra gain and dynamic range change on top of the automatic correction, if so desired by an operator.

Referring now to FIG. 3, a block diagram shows a process 53 for compression of image data that is implemented with the technique 40 (FIG. 2), such as by the image processor module 26 and RF processor module 22 (FIG. 1). In ultrasound systems, the data is typically compressed to reduce the data flow and display the image data in a scale that is easy to diagnose. According to an embodiment of the invention, the compression process 53 performed in technique 40 (FIG. 2) may be done in a non-linear fashion to incorporate all the dynamic range with different quantization scale at different gray scale level. According to one embodiment of the invention, at STEP 54, 8-bit raw data y is calculated (concurrent with the data acquisition of BLOCK 42, FIG. 2) by compressing the original linear data e in a nonlinear fashion according to:

y=f(e) [Eqn. 1],

where f( ) is the nonlinear compression function. Linear recompression of pre-compressed amplitudes y is then performed at STEP 56, according to:

z=g[f⁻¹(y)] [Eqn. 2],

where g( ) is the linear compression function, which may be a scaled logarithm compression. STEP 56 may be performed at during the estimation of BLOCK 46 (FIG. 2). Linear operations can then be performed to adjust gray scales at STEP 58 and the output is z2. Finally, the processed data is recompressed back to the nonlinear domain at STEP 60 according to:

y2=f[g⁻¹(z2)] [Eqn. 3].

STEPS 58 and 60 may be performed during the backend correction of BLOCK 52 (FIG. 2). Also, to save computation, the two re-compressions of STEPS 56 and 60 can be implemented using two look-up tables (LUTs).

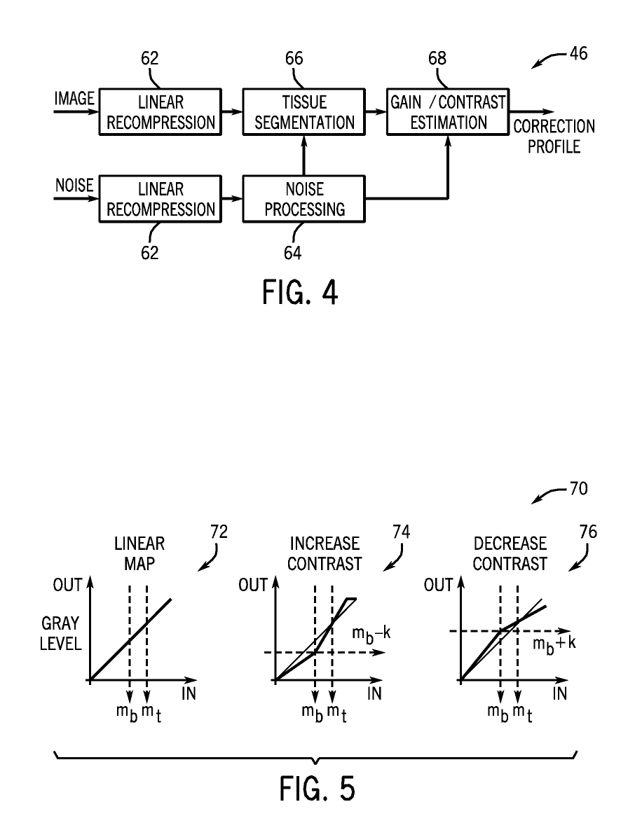

Referring now to FIG. 4, the estimation algorithm of BLOCK 46 (FIG. 2) is set forth in greater detail. As shown in FIG. 4, in a first step, the estimation algorithm takes image data and noise information as inputs and linearly recompresses the respective data at STEP 62. That is, as the image data and noise information is in the form of non-linear compressed data (i.e., the output y2 of STEP 60 in FIG. 3), a linear recompression is performed in order to convert the data to a linear log scale usable for quantitative estimation. With respect to the noise data input, the data can be either obtained using a noise model or by way of a noise measurement. The noise data is pre-processed before getting used, with the pre-processing being different for model data and measurement data. The outputs of the processing can be two smoothed noise curves along axial and lateral directions, respectively. The curves may be noted N(x) and N(y), where x and y are axial and lateral coordinates, respectively.

In embodiments in which a noise model is used, the outputs of the noise model may include the noise level in the axial direction. Optionally, the noise model may also include the noise level in the lateral direction. The information may be directly used as values N(x) and N(y) or be used after smoothing.

The noise data input can also include a noise measurement. The noise data measurement/input is linearly recompressed to a linear log scale at STEP 62 before processing, as set forth above. The data is processed at STEP 64, where assuming the axial and lateral noise data is separable, the noise frame is first averaged along both directions. The two averaged 1D vectors then go through a moving average for smoothing. The lateral vector is finally subtracted by its mean. Note the noise measurement is n(x,y), the noise model is N(x,y)=N(x)+N(y), and thus the processed noise level N(x) and N(y) can be described as:

$$\begin{matrix}N ( x ) = smooth ( \frac{\int n ( x , y ) d y}{\int d y} , {n}_{x} ) , & [ Eqn . 4 ] \\ and & \\ N ( y ) = smooth ( \frac{\int n ( x , y ) d x}{\int d x} , {n}_{y} ) - \frac{\int n ( x , y ) d x d y}{\int d x \int d y} , & [ Eqn . 5 ]\end{matrix}$$

where smooth( ) is a moving average function with kernel size of nₓ and n_{y} for axial and lateral directions, respectively. The kernel sizes are engineering defined as desired.

Referring still to FIG. 4, the linearly recompressed image data i(x,y) (from STEP 62) and the processed noise level N(x) and N(y) (from STEP 64) are input to STEP 66, where tissue segmentation is performed. At STEP 66, the noise level is first removed from the image data according to:

i₁(x,y)=i(x,y)−N(x)−N(y) [Eqn. 6],

and then tissue segmentation is performed for purposes of identifying or labeling dominant signals in image areas. According to one embodiment, amplitude thresholding may be used to segment tissue according to known techniques. The types of signals may be a tissue signal, bright signal, or noise signal, and the corresponding areas may be called a tissue area, bright area, and noise area, respectively. Different types of areas are treated differently in an estimation of gain and contrast performed at STEP 68.

Referring again to STEP 66, the image area is divided into 2D blocks or a 1D block if only TGC is to be corrected. The mean is evaluated for each block, such as by using the arithmetic average, and the maximal and minimal means for all the blocks, bₘₐₓ and bₘᵢₙ are then determined. The amplitude thresholds, b_{low}, and b_{high}, to separate noise, tissue, and bright interface are obtained by:

b_{low}=r_{low}(bₘₐₓ−bₘᵢₙ)+bₘᵢₙ [Eqn. 7],

and

b_{high}=r_{high}(bₘₐₓ−bₘᵢₙ)=bₘᵢₙ [Eqn. 8],

where r_{low} and r_{high} are percentage thresholds that separate the three different types of image areas (i.e., noise, tissue, and bright interface) and are engineering defined as desired.

Each block is marked as noise, tissue, or bright interface based on the block mean. If the block mean is smaller than b_{low}, then the block is a noise block. If the block mean is greater than b_{low} but smaller than b_{high}, then the block is a tissue block. If the block mean is greater than b_{high}, then the block is a bright block.

Referring still to STEP 66, the non-tissue blocks are re-evaluated. If some criteria are met, they may be re-marked as tissue blocks. STEP 66 thus serves to further identify weak tissue areas. Specifically, the mean of each non-tissue block is compared with its neighbor tissue block. If the mean difference is smaller than a pre-defined threshold, the block is re-identified as a tissue block. The process can run for several iterations, with the threshold and the number of iterations being engineering defined parameters.

At STEP 68, image gain and contrast estimation is performed. There are three methods for image gain estimation: TGC only, TGC and LGC, and 2D gain, which are similar to known techniques for gain estimation. A further simplified version of the methods is estimation of overall gain only, which can be easily done by evaluating average gain of the tissue region. In the TGC only method, each row of tissue blocks is evaluated to obtain an average gain at that row. Only center blocks are considered during the evaluation. The size of the center blocks is defined as the percentage of the total number of blocks in each row. The row is regarded as a valid tissue row only when the number of tissue blocks is over a certain percentage of the total number of the center blocks.

For each valid tissue row, the average gain is calculated by taking the arithmetic average of the valid tissue blocks in that row. Then, the average gain of non-tissue rows is calculated by interpolation and extrapolation from the gain of tissue rows.

For any non-tissue row that lies between valid tissue rows, linear interpolation is performed using two closest tissue rows, one at each side. For a non-tissue row that is outside the tissue row range, the average gain is the gain of the closest tissue row. Finally, a moving averaging is made on the TGC gain, or row-dependent gain.

The TGC gain is then propagated to 2D blocks for further processing:

g(r,c)=g(r) [Eqn. 9],

where g(r) is the row-dependent gain, g(r,c) is 2D block gain, or the row- and column-dependent gain, r is the row index, and c is the column index.

In the TGC and LGC method of gain estimation, average gain is calculated in both axial direction and lateral directions. The TGC gain is calculated in the same manner as the TGC only method set forth above. The LGC gain, or column-dependent gain, is calculated in a similar manner to the TGC gain, but with the dimension transposed. The LGC gain is noted by g(c) and the 2D block gain is calculated by:

g(r,c)=g(r)+g(c)−mean[g(c)] [Eqn. 10].

In the 2D gain method of estimation, thresholding is first made to the 2D block gains obtained in tissue segmentation. For any block with gain being less than the amplitude threshold b_{low}, the block gain is set to b_{low}; for any block with gain being greater than the amplitude threshold b_{high}, the block gain is set to b_{high}. Smoothing is then applied to the 2D block gains, with the smoothing being done first on each column in the axial direction, and then on each row in the lateral direction, using moving average.

After the 2D block gains are obtained, post-processing is performed. The post-processing including the steps of: (1) adding noise back to the block gain; (2) calculating the mean gain and adjusting the overall gain to a preset; (3) applying thresholding to limit the gain correction (the thresholding may be depth-dependent); (4) limiting the block gain so that noise level after correction is below a certain level; and (5) interpolating the block gain linearly to image samples. The calculated 2D gain profile is then used for gain correction.

In addition to the gain estimation/adjustment, image contrast can be estimated/adjusted at STEP 68 by controlling dynamic range, compression, and rejection. Similar to gain estimation, contrast estimation is performed on data after linear recompression as shown in FIG. 4. Automatic dynamic range adjustment, also called automatic contrast adjustment, can take input image data with or without gain correction and may be applied either before or after automatic gain adjustment to compensate dynamic range adaptively.

To measure the image contrast quantitatively, a contrast metric is developed. Tissue block segmentation is performed, as described in [Eqns. 6-8] so that the background region (i.e., non-tissue region) and tissue region are processed separately to calculate the image contrast. The percentage of the tissue region in the overall image frame gives additional information regarding the input image, such as whether the image is of a large tissue region, a cyst region, or a low penetration region, for example. Also, intensity difference between tissue and non-tissue regions aids in deciding the image contrast metric. The image contrast metric can be defined by:

Image contrast=(mₜ-m_{b})/m_{b} [Eqn. 11],

where mₜ is the average intensity of a tissue region and m_{b} is the average intensity of a non-tissue region. The expected contrast level is set based on the system characteristic, imaging target, and user preference. After the contrast of the image and the expected contrast level are compared, a decision can be made if image contrast needs to be increased or decreased.

Subsequent to a decision on whether image contrast should be increased or decreased, the image contrast may be controlled by one of two adaptive/dynamic contrast estimation methods or algorithms. The first adaptive dynamic contrast estimation method is an adaptive slope change method 70 as shown in FIG. 5. By the definition of the image contrast metric, this algorithm modifies the slope of an acquired gray mapping of the tissue region 72 by shifting the mean of the background region (m_{b}) down 74 (contrast increase) or up 76 (contrast decrease). The ratio between the contrast of the image (contrast_in) and the expected contrast level (contrast_model) can be used to calculate the mean shift (k) of the background region according to:

$$\begin{matrix}scale = \frac{contrast\_model}{contrast\_in} \\ = \frac{\frac{{m}_{t} - ( {m}_{b} ∓ k )}{( {m}_{b} ∓ k )}}{\frac{{m}_{t} - {m}_{b}}{{m}_{b}}} \\ = \frac{{m}_{b} ( {m}_{t} - {m}_{b} \pm k )}{( {m}_{b} ∓ k ) ( {m}_{t} - {m}_{b} )} .\end{matrix} \qquad [ Eqn . 12 ]$$

By replacing mₜ-m_{b} with diff, [Eqn. 9] can be reduced to

$$scale = \frac{{m}_{b} ( diff \pm k )}{( {m}_{b} ∓ k ) diff} . \qquad [ Eqn . 13 ]$$

Thus, the mean shift of the background is

$${k}_{inc / dec} = \frac{\pm {m}_{b} diff ( scale - 1 )}{{m}_{b} + diff scale} , \qquad [ Eqn . 14 ]$$

where k_{inc} represents the mean shift when the contrast needs to be increased and k_{dec} is the mean shift for the opposite case. Compared to a simple slope change method, the adaptive slope change method 70 avoids unwanted noise level increase or saturation.

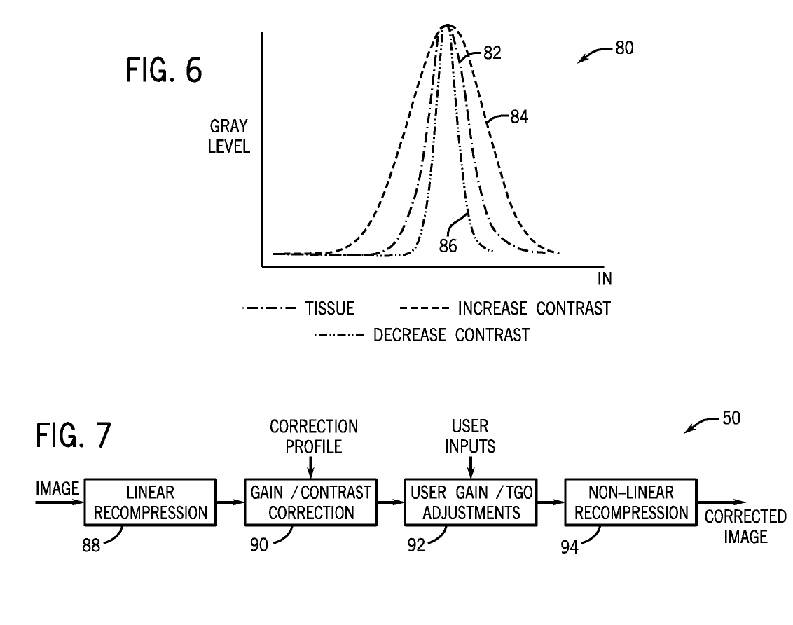

The second adaptive/dynamic contrast estimation method/algorithm is a tissue-specific histogram specification. A histogram specification is an image enhancement technique to modify the image so that it has a particular histogram as specified. As shown in FIG. 6, in the tissue-specific histogram specification method 80, the image contrast can be increased by remodeling the tissue histogram 82 to a broader distribution 84, or decreased by transforming the tissue histogram 82 to a narrower distribution 86, thereby modifying the slope of an acquired gray mapping. The tissue-specific histogram specification thus generates an adaptive gray mapping and is effective especially when tissue region is dominant.

The tissue-specific contrast estimation algorithm can be performed in 3 parts. In the first part, the histogram of the input image is found. Tissue blocks from the tissue segmentation performed at STEP 66 (FIG. 4) are employed to calculate a histogram from the tissue region only. Assuming that the input image has L pixel intensity levels lᵢ, i=0, 1, . . . , L-1, the tissue region of the input data to the contrast algorithm is x and the histogram of that region is hₓ. The probability function pₓ is given by:

$${p}_{x} ( {l}_{i} ) = \frac{{h}_{x} ( {l}_{i} )}{{\sum }_{i = 0}^{L - 1} {h}_{x} ( {l}_{i} )} , i = 0 , 1 , … , L - 1. \qquad [ Eqn . 15 ]$$

Then, the cumulative distribution Fₓ can be calculated by:

$${F}_{x} ( j ) = {\sum }_{i = 0}^{j} {p}_{x} ( {l}_{i} ) , \qquad [ Eqn . 16 ]$$

where j=0, 1, . . . , L-1.

In a second part of the tissue-specific contrast estimation algorithm, a desired histogram is specified to modify the acquired tissue histogram to a desired histogram model. Rician distribution is an appropriate model in echocardiography, but Gaussian distribution is used to reduce complexity. With a desired mean (μ) and standard deviation (σ), the Gaussian distribution model p_{d} is:

$${p}_{d} ( {l}_{i} ) = \frac{1}{σ \sqrt{2 π}} {e}^{- {( {l}_{i} - ϖ )}^{2} / ( 2 {σ}^{2} )} . \qquad [ Eqn . 17 ]$$

A cumulative distribution

$${F}_{d} ( j ) = {\sum }_{i = 0}^{j} {p}_{d} ( {l}_{i} )$$

is then calculated from the histogram of the Gaussian model.

In a third part of the tissue-specific contrast estimation algorithm, for each gray level lᵢ, Fₓ(lᵢ) is found, and then a j level is found so that F_{d}(j) best matches Fₓ(lᵢ). A lookup table can thus be built according to:

|Fₓ(lᵢ)−F_{d}(j)|=minₖ|Fₓ(lᵢ)−F_{d}(k)| [Eqn. 18].

Thus, the new lookup table m is

m(lᵢ)=j [Eqn. 19].

In both contrast adjustment approaches, i.e., the adaptive slope change method and the tissue-specific histogram specification method, the mean of the tissue region (mₜ) is not altered to keep the gain level the same.

It is recognized that the contrast adjustment approaches described above and shown in FIGS. 5-6 can be applied based, in part, on the anatomy of the volume being imaged. That is, the adaptive slop change method for contrast adjustment is suitable for imaging targets where cavities are presented, like cardiac applications, whereas the tissue-specific histogram specification method is suitable for imaging targets where the main component is tissue, like general abdomen applications.

The contrast adjustment approaches may be applied on the overall image and generate one lookup table to adjust the whole image. Alternatively, localized contrast adjustments may be implemented by dividing the image into sub-images and applying the same methods to each sub-image. In this case, each region in the image has its own lookup table. To eliminate transition artifacts, spatial smoothing may be applied to the lookup tables before contrast adjustment is made.

Referring now to FIG. 7, the compensation/correction algorithm of BLOCK 50 (FIG. 2) is set forth in greater detail. Like the estimation algorithm, the correction algorithm first performs a linear recompression on the image data at STEP 88. Upon linear recompression, the correction processing is performed in two parts, with gain/contrast correction performed at STEP 90 and user adjustments performed at STEP 92. As shown in FIG. 2, the gain/contrast correction performed at STEP 90 and the user adjustments performed at STEP 92 can be applied as both a front-end correction and a back-end correction (i.e., applied to front-end electronics 25 and back-end electronics 33 of ultrasound system 10, FIG. 1). Referring again to FIG. 7, after the correction processing, non-linear recompression is performed at STEP 94 to output a corrected image.

With respect to gain correction performed at STEP 90, the 2D gain profile is simply added to the image to obtain a corrected image according to:

i₁(x,y)=i(x,y)+g(x,y) [Eqn. 20],

where i is the linearly recompressed image frame, g is the 2D gain profile, and i₁ is the corrected image.

With respect to contrast correction performed at STEP 90, since the new gray map is calculated from the contrast estimation, contrast correction entails simply applying the new lookup table m to the input image according to:

i₂(x,y)=m(i(x,y)) [Eqn. 21],

where i is the linearly recompressed image frame, and i₂ is the contrast adjusted image.

According to an embodiment of the invention, the operator can impose manual gain/TGC adjustments at STEP 92 on top of the automatic correction. The user gain and TGC values are relative changes to when the automatic correction (i.e., STEP 92) is activated. The user adjustments may be expressed by:

i₄(x,y)=i₃(x,y)+Δgᵤ+Δtgcᵤ(x) [Eqn. 22],

where Δgᵤ and Δtgcᵤ are the user gain increment and user TGC increment, respectively.

As set forth above, prior art existing auto optimization features have typically been implemented in a static mode, in which the function is activated by the user through a button or other means of user interface. The estimation process is then run for the incoming frame or set of frames to obtain a compensational profile. This fixed profile is then applied to the subsequent frames until the user manually activates another estimation. Existing semi-dynamic features also have been previously implemented in the art, but such features only activate a new estimation when the gain difference is beyond a threshold. Thus, the optimization is not continuous and smooth, resulting in a user suffering from untimely step changes. Embodiments of the invention overcome the aforementioned drawbacks of static and semi-dynamic features by providing real-time, dynamically optimized gain and contrast in tracking and compensation during ultrasound imaging.

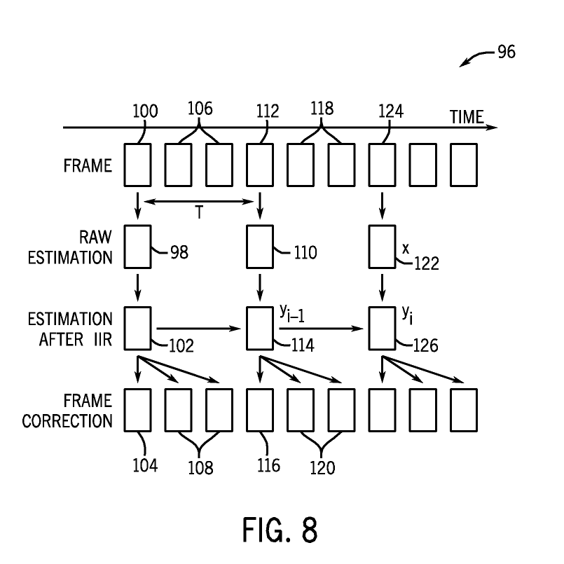

According to embodiments of the invention, the compensation/correction profile generated by image processor module 26 (FIG. 1) is tracked and updated in real-time or near real-time to reflect the time-varying change in tissue attenuation. FIG. 8 is a block diagram illustrating the compensational process 96 according to an embodiment of the invention. As shown in FIG. 8, a first compensation/correction profile 98 is estimated upon acquisition of a first image frame data set 100. The raw estimation from a single frame or set of frames is then further processed using an infinite impulse response (IIR) filter to produce a filtered compensation/correction profile 102. The filter can be expressed:

yᵢ=cxᵢ+(1−c)yᵢ₋₁ [Eqn. 23],

where xᵢ is the current raw estimation, yᵢ is the current processed estimation, yᵢ₋₁ is the previously processed estimation, and c is the IIR coefficient that is engineering defined. The coefficient c provides a trade-off between responsiveness and smoothness. Alternatively, other forms of filters, like a finite impulse response (FIR) filter, may be used. The FIR filter provides similar performance but requires more memory space for processing. The purpose of applying a smoothing filter is to reduce estimation errors from each frame and hence improve the performance.

The filtered compensation/correction profile 102 is then applied to the first image frame data set to generate an optimized image frame data set 104 having improved gain and contrast. As shown in FIG. 8, additional image frame data sets 106 are acquired subsequent to acquisition of the first image frame data set 100. According to one embodiment of the invention, no compensation/correction profile is estimated for the additional image frame data sets 106. Instead, the previously estimated and filtered compensation/correction profile 102, is applied to the additional image frame data sets 106 to produce optimized image frame data sets 108. After pre-determined time interval T has passed, another compensation/correction profile 110 (i.e., a modified compensation/correction profile) is estimated. The modified compensation/correction profile 110 can be estimated from a single image frame data set 112. Alternatively, it is recognized that the modified compensation/correction profile 110 could be estimated from image frame data set 112 and from image frame data sets 106, according to another embodiment of the invention. The modified compensation/correction profile 110 is further processed using an IIR filter to produce a filtered modified compensation/correction profile 114, which is then applied to image frame data set 112 to generate an optimized image frame data set 116 having improved gain and contrast. As shown in FIG. 8, the filtered modified compensation/correction profile 114 is then applied to subsequently acquired image frame sets 118 to generate additional optimized image frame data sets 120, until estimation of a next compensation/correction profile 122 from an image frame data set 124 after the pre-determined time interval T has again passed. The time interval T may be chosen to be a value small enough, for example, 50 milliseconds, so that a user's experience of delay and un-smoothness is minimal. Thus, as shown in the embodiment of FIG. 8, an estimation of a compensation/correction profile is not done on every image frame data set in order to reduce the amount of computation performed by processor module 26 (FIG. 1).

With respect to the automatic transition from one compensation/correction profile to the next compensation/correction profile, such as the transition between profiles 110 and 122, it is recognized that each profile is estimated from single frames taken some time apart (i.e., frames 112 and 124). Thus, according to an embodiment of the invention, the compensation/correction profile 110 estimated and applied from frame 112, for example, can be gradually shifted to produce “shifted correction profiles.” That is, for each frame 118 between the frames 112, 124 used for estimation of the correction profiles 110, 122 the gain and contrast parameters that are applied are adjusted slightly. According to an embodiment of the invention, prediction or extrapolation can be implemented between the estimation of the correction profiles 110, 122 that is based on two or more previously estimated compensation/correction profiles, such as compensation/correction profiles 102, 114 and any other previously estimated compensation/correction profiles. The prediction or extrapolation can be linear or higher-order. The shifted correction profile(s) applied to the frames 118 would thus gradually be adjusted such that all frames acquired between the frames 112, 124 are compensated by correction profiles that transition gradually from the compensation/correction profile 114 to the compensation/correction profile 126, thereby generating optimized image frame data sets 120 having gradually shifted gain/contrast settings such that no abrupt change in image settings is experienced. According to another embodiment of the invention, in a recalled mode where a recorded image loop is replayed, since correction profiles can be pre-calculated, the shifted correction profiles for frames 118 can be simply calculated using linear interpolation or higher-order interpolation.

While the compensational process 96 of FIG. 8 is shown as implementing a pre-determined time interval T between estimation of the compensation/correction profiles 98, 110, 122, it is recognized that, according to alternative embodiments of the invention, estimation of a compensation/correction profile could be done on every image frame data set 100, 106, 112, 118 using the gain and contrast estimated methods shown/described with respect to FIGS. 4-7. Each estimated compensation/correction profile would then be applied to its corresponding image frame data set to produce an image frame data set having optimized gain and contrast.

A technical contribution for the disclosed method and apparatus is that it provides for a computer implemented system and method for dynamic optimization of gain and contrast in a real-time fashion for ultrasound imaging.

Therefore, according to one embodiment of the invention, an ultrasound system includes a transducer array comprising a multiplicity of transducer elements configured to acquire image frame data sets of raw acoustic data samples during ultrasound scanning of an imaging volume and a display system for displaying an image frame data set. The ultrasound system also includes an image processor module programmed to dynamically estimate a correction profile in real-time and apply the correction profile to adjust a gain and contrast of the image frame data sets. In being programmed to dynamically estimate the correction profile, the image processor module is programmed to identify a tissue region and a background region in an image frame data set, determine an image intensity for each of the tissue region and the background region, and formulate a gain profile based on the image intensity of the tissue region to compensate the gain variation of an image displayed on the display system. The image processor module is further programmed to calculate an image contrast metric based on the image intensity of the tissue region and the image intensity of the background region and modify a gray map of the image frame data set based on the image contrast metric to adjust the contrast of an image displayed on the display system.

According to another embodiment of the invention, a method for dynamically optimizing gain and contrast in ultrasound imaging during acquisition of a plurality of frames of ultrasound image data includes the steps of segmenting a frame of ultrasound image data into a plurality of tissue blocks and a plurality of non-tissue blocks and determining an image intensity for each of the plurality of tissue blocks and each of the plurality of non-tissue blocks. The method further includes the steps of estimating a gain correction for the frame of ultrasound image based on the segmented plurality of tissue blocks and the plurality of non-tissue blocks, estimating a contrast correction based on the image intensity of the plurality of tissue blocks and the image intensity of the plurality of non-tissue blocks, and applying a correction profile to the frame of ultrasound image data based on the estimated gain correction and the estimated contrast correction.

According to yet another embodiment of the invention, an ultrasound system includes a transducer array comprising a multiplicity of transducer elements configured to acquire frames of ultrasound image data during ultrasound scanning of an imaging volume. The ultrasound system also includes an image processor module operationally connected to the transducer array to receive the frames of ultrasound image data therefrom, wherein the image processor module is programmed to: (a) segment a first frame of ultrasound image data into a plurality of tissue blocks and a plurality of non-tissue blocks, (b) determine an image intensity for each of the plurality of tissue blocks and each of the plurality of non-tissue blocks, and (c) estimate a gain correction for the first frame of ultrasound image based on the segmented plurality of tissue blocks and the plurality of non-tissue blocks. The image processor module is further programmed to: (d) estimate a contrast correction for the first frame of ultrasound image based on the image intensity of the plurality of tissue blocks and the image intensity of the plurality of non-tissue blocks, (e) apply the estimated gain correction and the estimated contrast correction to the first frame of ultrasound image data, and (f) estimate a modified gain correction and a modified contrast correction for a next frame of ultrasound image data according to steps (a)-(d), the modified gain correction and the modified contrast correction being applied to the next frame of ultrasound image data.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasound system comprising:
a transducer array comprising a multiplicity of transducer elements configured to acquire image frame data sets of raw acoustic data samples during ultrasound scanning of an imaging volume;
a display system for displaying an image frame data set; and
an image processor module programmed to dynamically estimate a correction profile in real-time and apply the correction profile to adjust a gain and contrast of the image frame data sets, wherein the processor, in being programmed to dynamically estimate the correction profile, is programmed to:
identify a tissue region and a background region in an image frame data set;
determine an image intensity for each of the tissue region and the background region;
formulate a gain profile based on the image intensity of the tissue region to compensate the gain variation of an image displayed on the display system;
calculate an image contrast metric based on the image intensity of the tissue region and the image intensity of the background region; and
modify a gray map of the image frame data set based on the image contrast metric to adjust the contrast of an image displayed on the display system.

2. The ultrasound system of claim 1 wherein the image processor module is further programmed to:
determine a tissue histogram of the tissue region of the image frame data set; and
modify a distribution of the tissue histogram to form a corrected histogram, the corrected histogram corresponding to the modified gray map of the image frame data set.

3. The ultrasound system of claim 2 wherein the image processor module is further programmed to build a lookup table from the tissue histogram and the corrected histogram.

4. The ultrasound system of claim 1 wherein the image processor module is further programmed to:
determine a mean image intensity of the tissue region; and
determine a mean image intensity of the background region.

5. The ultrasound system of claim 4 wherein the image processor module is further programmed to shift the mean image intensity of the background region to modify the gray map of the tissue region.

6. The ultrasound system of claim 4 wherein the image processor module is further programmed to maintain the mean image intensity of the tissue region at a constant value.

7. The ultrasound system of claim 1 wherein the image processor module is further programmed to process the estimated correction profile with a filter, the filter comprising one of an infinite impulse response (IIR) filter and a finite impulse response (FIR).

8. The ultrasound system of claim 1 wherein the image processor module is further programmed to:
estimate a first correction profile for a first image frame data set;
apply the first correction profile to the first image frame data set and to at least one additional image frame data set acquired subsequent to the first image frame data set; and
estimate a second correction profile for an image frame data set acquired subsequent to the at least one additional image frame data set.

9. The ultrasound system of claim 8 wherein the image processor module is further programmed to temporally separate estimation of the second correction profile and estimation of the first correction profile by a pre-determined time interval, the pre-determined time interval encompassing acquisition of the at least one additional frame of ultrasound image data.

10. The ultrasound system of claim 8 wherein the image processor module is further programmed to:
apply at least one shift to the first correction profile to generate at least one shifted correction profile; and
apply the at least one shifted correction profile to the at least one additional frame of ultrasound image data;
wherein the at least one shifted correction profile transitions between the first correction profile and the second correction profile.

11. The ultrasound system of claim 1 wherein the image processor module is further programmed to dynamically estimate a correction profile for each acquired image frame data set.

12. The ultrasound system of claim 1 wherein the image processor module is further programmed to estimate at least one of an overall gain compensation, a time gain compensation, a lateral gain compensation, and a 2D gain.

13. A method for dynamically optimizing gain and contrast in ultrasound imaging during acquisition of a plurality of frames of ultrasound image data, the method comprising:
segmenting a frame of ultrasound image data into a plurality of tissue blocks and a plurality of non-tissue blocks;
determining an image intensity for each of the plurality of tissue blocks and each of the plurality of non-tissue blocks;
estimating a gain correction for the frame of ultrasound image based on the segmented plurality of tissue blocks and the plurality of non-tissue blocks;
estimating a contrast correction based on the image intensity of the plurality of tissue blocks and the image intensity of the plurality of non-tissue blocks; and
applying a correction profile to the frame of ultrasound image data based on the estimated gain correction and the estimated contrast correction.

14. The method of claim 13 further comprising updating the estimated gain correction and the estimated contrast correction during acquisition of the plurality of frames of ultrasound image data.

15. The method of claim 14 wherein updating the estimated gain correction and the estimated contrast correction comprises:
estimating a first gain correction and a first contrast correction for a first frame of ultrasound image data in the plurality of frames of ultrasound image data;
instituting a pre-determined time delay upon estimation of the first gain correction and the first contrast correction for the first frame of ultrasound image data, wherein acquisition of at least one additional frame of ultrasound image data occurs during the pre-determined time delay;
estimating a modified gain correction and a modified contrast correction for another frame of ultrasound image data in the plurality of frames of ultrasound image data after expiration of the pre-determined time delay.

16. The method of claim 15 further comprising:
applying a correction profile to the first frame of ultrasound image data and the at least one additional frame of ultrasound image data based on the first gain correction and the first contrast correction; and
applying a modified correction profile to the another frame of ultrasound image data upon estimation of the modified gain correction and the modified contrast correction.

17. The method of claim 13 wherein estimating the contrast correction comprises:
calculating a tissue histogram from the segmented tissue blocks; and
modifying a distribution of the tissue histogram to form a corrected histogram.

18. The method of claim 13 wherein estimating the contrast correction comprises:
determining a mean image intensity of the tissue blocks;
determining a mean image intensity of the non-tissue blocks; and
shifting the mean image intensity of the non-tissue blocks based on a comparison of the mean image intensity of the tissue blocks and the mean image intensity of the non-tissue blocks.

19. The method of claim 13 further comprising processing the estimated gain correction and the estimated contrast correction with a filter to produce a smoothed correction profile.

20. An ultrasound system comprising:
a transducer array comprising a multiplicity of transducer elements configured to acquire frames of ultrasound image data during ultrasound scanning of an imaging volume; and
an image processor module operationally connected to the transducer array to receive the frames of ultrasound image data therefrom, wherein the image processor module is programmed to:
(a) segment a first frame of ultrasound image data into a plurality of tissue blocks and a plurality of non-tissue blocks;
(b) determine an image intensity for each of the plurality of tissue blocks and each of the plurality of non-tissue blocks;
(c) estimate a gain correction for the first frame of ultrasound image based on the segmented plurality of tissue blocks and the plurality of non-tissue blocks;
(d) estimate a contrast correction for the first frame of ultrasound image based on the image intensity of the plurality of tissue blocks and the image intensity of the plurality of non-tissue blocks;
(e) apply the estimated gain correction and the estimated contrast correction to the first frame of ultrasound image data; and
(f) estimate a modified gain correction and a modified contrast correction for a next frame of ultrasound image data according to steps (a)-(d), the modified gain correction and the modified contrast correction being applied to the next frame of ultrasound image data.

21. The ultrasound system of claim 20 wherein the processor module is further programmed to estimate a gain correction for the frame of ultrasound image based on the segmented plurality of tissue blocks and the plurality of non-tissue blocks.

22. The ultrasound system of claim 20 wherein the image processor module is further programmed to:
calculate a tissue histogram from the segmented tissue blocks; and
modify a distribution of the tissue histogram to form a corrected histogram.

23. The ultrasound system of claim 20 wherein the image processor module is further programmed to:
determine a mean image intensity of the tissue blocks;
determine a mean image intensity of the non-tissue blocks; and
shift the mean image intensity of the non-tissue blocks based on a comparison of the mean image intensity of the tissue blocks and the mean image intensity of the non-tissue blocks.

24. The ultrasound system of claim 20 wherein the image processor module is further programmed to:
apply a pre-determined time delay between estimation of the gain and contrast correction for the first frame of ultrasound image and the estimation of the modified gain and contrast correction for the next frame of ultrasound image data; and
apply the estimated gain and contrast correction to at least one additional frame of ultrasound image data acquired during the pre-determined time delay.

25. The ultrasound system of claim 20 wherein the image processor module is further programmed to apply the estimated gain corrections and the estimated contrast corrections to front-end hardware and back-end electronics in the ultrasound system.
